SEQUENCE LISTING (45 sequences)

SEQ ID NO: 1 - DNA, 1539 bp, Homo sapiens
  CDS (1)..(1539): Tieg2wt
atgcacacgccggacttcgcaggcccagacgacgcgcgcgcagttgacatcatggacatatgtgagtccatcctggagaggaagcggcatgacagcgaaaggtctacttgcagcatcttggagcagacagacatggaagctgtcgaggctcttgtttgtatgagctcctggggtcaaagatcccagaaaggtgacctgttgcggataagacccctcacgcctgtctctgactctggggatgtcaccaccactgtgcatatggatgcagccacacctgaactaccaaaagacttccattctttatcgactctgtgcataactcctcctcagagccctgatctcgtggagccatcgacaaggacacctgtttctccccaagtaacagattccaaagcatgtacagccacggatgttctccagtcctctgccgtagtggccagagctctgagcgggggcgcggagaggggcttgctgggtttggagccagtgcccagctctccctgcagggccaaggggactagcgtgatccgacacactggggagagccctgctgcctgctttcccaccatccagactccagattgccggctttctgacagcagagaaggagaagagcagcttctgggacactttgaaactttgcaggacacacacctcacggacagtttactcagcactaacttggtgtcctgtcagccctgcttgcacaagtctggtggcctgctgctcactgacaaaggccagcaggcagggtggcctggtgcagttcagacttgctcaccaaagaattatgaaaatgacctgcccaggaaaaccacccctctgatttctgtctctgtccctgctccccctgtcctttgccagatgatccctgtgactggacaaagtagcatgttaccagcttttttgaagccccctccccagttgtctgtggggactgtgagacccatcctagctcaggctgctccagcgcctcaacctgtgttcgtgggacctgctgtgcctcagggagctgtgatgttggtcctgccccagggagccctccctccgcctgccccctgtgcagccaatgtcatggctgccgggaataccaagttgttgccccttgcccctgctccagtgttcatcacctctagccaaaactgtgtccctcaggtagacttttcccgaaggaggaactatgtttgcagcttcccaggttgccggaagacctacttcaaaagttcccaccttaaggcccatcttcgcactcacacaggggagaagcctttcaactgcagctgggatggctgtgataaaaagtttgctcgttcggatgagctgtcacgccaccgcagaactcacacaggggagaagaagtttgtgtgcccggtgtgtgaccgacgtttcatgcgcagtgaccacctgacgaagcatgcccggcgccacatgacgaccaagaagatcccaggctggcaggcagaggttggcaagctgaacagaatcgcctctgcagagagcccggggagcccactggtgagcatgccagcctctgcctga

SEQ ID NO: 2 - DNA, 267 bp, Homo sapiens
  Exon 2 tieg2 gene
gttgacatcatggacatatgtgagtccatcctggagaggaagcggcatgacagcgaaaggtctacttgcagcatcttggagcagacagacatggaagctgtcgaggctcttgtttgtatgagctcctggggtcaaagatcccagaaaggtgacctgttgcggataagacccctcacgcctgtctctgactctggggatgtcaccaccactgtgcatatggatgcagccacacctgaactaccaaaagacttccattctttatcgact

SEQ ID NO: 3 - protein, 512 aa, Homo sapiens
  TIEG2 Q62R
MHTPDFAGPDDARAVDIMDICESILERKRHDSERSTCSILEQTDMEAVEALVCMSSWGQRSRKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLTDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGALPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

SEQ ID NO: 4 - DNA, 2818 bp, Homo sapiens
  -1.8 kb of TIEG2
gactggaggtcggccccgagggtgtgtccgctagagggggcctctccaggtggactgcgggtgagagtaaagggggcatgtgggggaaaagcattctagggaacagcaagtgcgaggacctggagacggcagaggggctggtgggacaggcgcccaagaggaggcccaggctgtccctccggggcgtggggagaggcatggggctgggtccctgcaggagctcacaagccacagtggggagcagaagcgttacgctgagtacagtgggagccactgaaagggttaaagacccctgggggtgctgagtgggaagaggctctgggagctggagtggacgcagggagaccagttataaggcgaatgcaggagtccaggcaaaagagaatattccagtgtgtatcttaagacttaactttttacacacagaacaccctgcgcccctctcagagcctccggggtcttcatgcgagcagtttatcacggttctttcgtcttaaaccctcttactatttcagcaaacaccctctcctcagcctggtcaacccctctgcccaacccgagtgggccccgaaggaacaagtggggaccagagtcctgctcatgtgagtggtggggcccagcccgttcccctaagaccagaattctctcgctcagagcaaagccccggggtgccgggattctctctgctcccgctccctggatggagcttttaaaaaaggaggattcctcactgcggcctctgccccttgcactgagcgccaacccgcatcacagctcagcaaaggcgcgcgcagtgaacagacggaggaggggtggctcctcgctgagctgaaggctgcggcgacgaaaataaacagcatggttcctgctctccacgaacctggcgctgcagacgtgggcaaaccacaccaagagacgcgagcaaccgaaatatagggagaaaaaaaaaggtgccctggacagcgggctagatgtctcctgcaggctcgaggacttcccggagggggcggcgttttctcggggcctgggaagaatgggcgggtttcagcgggcgccttctgggtgactgcccgtttcggcttcccctgaccccgtaacagagcggaagaggcccagagacgccagaggaggggcgtcagcgggacactcaggccaataagctctgactcccgctgtctgcaaaaatgaccggatgcaggaatgtcacctagaaccggccagctttccgacctctccggtcaaggacgcattcctaagagacgaagggagaatgacagaagttaaaaacaaccaaacaaaagccaggttgaggcctctaggtgggtctccgctaccgatggattcagccggtggggctgcaggaggttcctataagcgtggtcccctctccccgcaccgagtccggccccggccctgggatttccccagccgccgccccgccccctccgccccgccgcccccgccgccccgccgccccctccgcccccgccgccccctccgcccccgccgccccctccgccccgcttgcccctggcgccgctgcgacctttcccttactttccagggcctcaggccccgcccctcgctccgaccaaaacacgtgccgcgtcccgccctgcccgatcacgccgcggccaggctccgatcacgcggccccccgcggcgctcattggccggccgggcacgaattggctggtggtggccggccccgccccggcaggggcgcggtgtattttggttcgcctccgcgccccgccccgccagccgctccctccgcggccgccccgcccctcccgcgccgcgagggccgcgccggggcagagccgcgcgggcgggcgaggcgcgtgccggccgcaggagctccgggttgccgccgccgccgccgcccgcagcccacgtgcggccgctgctgcgcccgagctcacgccccgcggccgctttgttgctcccggccggcctgcacgatgcacacgccggacttcgcaggcccagacgacgcgcgcgcagtgagtggtggggctgccgcggcgggactactcgtctgagcgaggggcgaggcgggggaagtggtgcggcactcgcgcgcctgtaaatgcgggaggtggggcgtgcagggcttcgctgcggccgcgccggtaggggcctgggcgggctccggagccgggcggggcggcggccgcgacgggcgcgcccgggtcggcgggggcgaggagggggcgtgttccccgcgcagctttgtcttgcgcttcctgggcggccccgccccgctggccccgcggctatttccagccatgacgtcaccccggtcctgtgagccggacggccgttgggcgggggagcggctggcgggaacgcggcacgcggccttgggggcggggcaagagctgctggcgggaacgcggcacggttttgggggcggggcacgcggccgtcgggcgggttaagagcggctagcggtagtgccgctcgggcctgggggcgggcaaggtctaggggccggggcaacgacgggaggcgggagcgccgcactgccttggaggcggggcaacgcttggaggcgggaacgcggcacgcgagcgttgggggccctaagcgtcgcgacctgggggcttcggggaggtagccgtggacgtgggcagagcgcgggcctcgtggtgtgcgggcactgtgggccgggcgagccccttcccgcccgtgtggtgagtcggcctcggcgcccggttctgtgacatcacagggggtttagtggcgcagcctgcgggacagaggccggggatttgaggtggcctcgtcttgtttgatctcggggaacctcggc

SEQ ID NO: 5 - DNA, 21 bp, Artificial Sequence
  Description of Artificial Sequence Primer
caaagatcccrgaaaggtgac

SEQ ID NO: 6 - DNA, 21 bp, Artificial Sequence
  Description of Artificial Sequence Primer
ggggtgctgastgggaagagg

SEQ ID NO: 7 - DNA, 23 bp, Artificial Sequence
  Description of Artificial Sequence Primer of exon 2 TIEG2 A185G variant
ctcggtgtttgttgctatagact

SEQ ID NO: 8 - DNA, 23 bp, Artificial Sequence
  Description of Artificial Sequence Primer of exon 2 TIEG2 A185G variant
cagggaatcttctcacaagttct

SEQ ID NO: 9 - DNA, 24 bp, Artificial Sequence
  Description of Artificial Sequence Primer for the sequence of TIEG2 G-1534C variant
gtttaagacgaaagaaccgtgata

SEQ ID NO: 10 - DNA, 19 bp, Artificial Sequence
  Description of Artificial Sequence Primer for the sequence of TIEG2 G-1534C variant
gaacagcaagtgcgaggac

SEQ ID NO: 11 - DNA, 24 bp, Artificial Sequence
  Description of Artificial Sequence Primer SNP436
ggaaccatgctgtttattttcgtc

SEQ ID NO: 12 - DNA, 24 bp, Artificial Sequence
  Description of Artificial Sequence Primer SNP436
gtcttcatgcgagcagtttatcac

SEQ ID NO: 13 - DNA, 21 bp, Artificial Sequence
  Description of Artificial Sequence Primer SNP442
gagacgcgagcaaccgaaata

SEQ ID NO: 14 - DNA, 24 bp, Artificial Sequence
  Description of Artificial Sequence Primer SNP442
agaggggaccacgcttataggaac

SEQ ID NO: 15 - DNA, 22 bp, Artificial Sequence
  Description of Artificial Sequence Primer SNP453
aaacaaccaaacaaaagccagg

SEQ ID NO: 16 - DNA, 22 bp, Artificial Sequence
  Description of Artificial Sequence Primer SNP453
ggtgtattttggttcgcctccg

SEQ ID NO: 17 - DNA, 23 bp, Artificial Sequence
  Description of Artificial Sequence Primer SNP451
gggcacgaattggctggtggtgg

SEQ ID NO: 18 - DNA, 21 bp, Artificial Sequence
  Description of Artificial Sequence Primer SNP451
gcgggactactcgtctgagcg

SEQ ID NO: 19 - DNA, 20 bp, Artificial Sequence
  Description of Artificial Sequence Primer SNP444
ttcatatgcgtcttggtcat

SEQ ID NO: 20 - DNA, 19 bp, Artificial Sequence
  Description of Artificial Sequence Primer SNP444
cgctcagacgagtagtccc

SEQ ID NO: 21 - DNA, 20 bp, Artificial Sequence
  Description of Artificial Sequence Primer SNP417
acaggtgtccttgtcgatgg

SEQ ID NO: 22 - DNA, 24 bp, Artificial Sequence
  Description of Artificial Sequence Primer SNP417
agaaagcagatttggagagggata

SEQ ID NO: 23 - DNA, 17 bp, Artificial Sequence
  Description of Artificial Sequence Primer SNP305
atctcgtggagccatcg

SEQ ID NO: 24 - DNA, 20 bp, Artificial Sequence
  Description of Artificial Sequence Primer SNP305
acagaaatcagaggggtggt

SEQ ID NO: 25 - DNA, 18 bp, Artificial Sequence
  Description of Artificial Sequence Primer SNP306
ggcctggtgcagttcaga

SEQ ID NO: 26 - DNA, 20 bp, Artificial Sequence
  Description of Artificial Sequence Primer SNP306
gaagatgggccttaaggtgg

SEQ ID NO: 27 - DNA, 23 bp, Artificial Sequence
  Description of Artificial Sequence Primer SNP416
acgtctactgcaatgtgaacaga

SEQ ID NO: 28 - DNA, 24 bp, Artificial Sequence
  Description of Artificial Sequence Primer SNP416
cctacttcaaaagttcccacctta

SEQ ID NO: 29 - DNA, 22 bp, Artificial Sequence
  Description of Artificial Sequence Primer SNP300
tttcctttctctttaatatgta

SEQ ID NO: 30 - DNA, 17 bp, Artificial Sequence
  Description of Artificial Sequence Primer SNP300
gaggaatccatcagttc

SEQ ID NO: 31 - DNA, 24 bp, Artificial Sequence
  Description of Artificial Sequence Primer SNP418
aaaagaaggctgcaaaatgatcta

SEQ ID NO: 32 - DNA, 20 bp, Artificial Sequence
  Description of Artificial Sequence Primer SNP418
cctctgcctgaaaggtccat

SEQ ID NO: 33 - DNA, 16 bp, Artificial Sequence
  Description of Artificial Sequence Primer for the sequence of TIEG2 G-1534C variant
tcttcccactcagcac

SEQ ID NO: 34 - DNA, 26 bp, Artificial Sequence
  Description of Artificial Sequence Primer for the sequence of TIEG2 G-1534C variant
tccctgcgtccactccagctcccaga

SEQ ID NO: 35 - DNA, 18 bp, Artificial Sequence
  Description of Artificial Sequence Primer of exon 2 TIEG2 A185G variant
atcccagaaaggtgacct

SEQ ID NO: 36 - DNA, 27 bp, Artificial Sequence
  Description of Artificial Sequence Primer of exon 2 TIEG2 A185G variant
tcttgtttgtatgagctcctggggtca

SEQ ID NO: 37 - DNA, 402 bp, Homo sapiens
  Variant G-1534C (SNP437b)
gaacagcaagtgcgaggacctggagacggcagaggggctggtgggacaggcgcccaagaggaggcccaggctgtccctccggggcgtggggagaggcatggggctgggtccctgcaggagctcacaagccacagtggggagcagaagcgttacgctgagtacagtgggagccactgaaagggtaaaaacccctgggggtgctgastgggaagaggctctgggagctggagtggacgcagggagaccagttataaggcgaatgcaggagtccaggcaaaagagaatattccagtgtgtatcttaagacttaacacttatttttacacacagaacaccctgcgcccctctcagagcctccggggtcttcatgcgagcagtttatcacggttctttcgtcttaaa

SEQ ID NO: 38 - DNA, 446 bp, Homo sapiens
  Sequence of 304a
gaaggaggctccgtaatcagaccttttgtgtggtgagggtctggaccccctggggacaaatcggactttctaaggtgggcaggggctgtgttttctgaggacatatggcagcctttagttttaagagttgggaaggaaaagaaggaagagtgaagcctgtggctccatgatgtactttgcaatcctgggccaggggtggaaggaaataatggaagacttccaagcacttcttccagagtagctggaaaaccactttcaattctacctctttgcagccaccccctggcacaggaagggcacgagcactgacggccacagggtcacccggcacactactgggtctgtctggaccagacccactgccagagtggccacttcacatgttggctttttgagacagcgtcttgctctgttgcccaggctagggtgcagtggtatgatcatag

SEQ ID NO: 39 - DNA, 1132 bp, Homo sapiens
  Sequence of 54a (SHGC24187)
aacagcgcggcttctgctgaagccagtacaagcctggttatctggcttgacccgcccacacggaggcctccccagcgttttctttttcccgagaagagctccagctcgcaggtccttagaacacggcagagcaggagcatctcaggaaccaacggccagctccttggcgctgtccccagccccacaggcctgctgcggccatccgcaaacgggcatttccttgaacttttgagagtgcctttgaccagctgttcgcagctgggaaactccaacttttgaagtcagagcttggagcggcccccgcaagggcgtggctcaaaccctccccagagctgggagccattgcttatgcaagcccgtttcttttgatgtaaagatggaagtcgttactatattttaataatgcaaatacatcttttaaaataagttaacatttcttaccaccagatggatcaggctttgaatttaatcatgtaaatgtttgtaattttatgtcattttgttaaaatgggacgctttcaattggtttccaagaaagatgatacctctgcattttctggtggaaaaggtgtaatacccttaatgagatcaaagtgttaggggaaaaaaattccaaaagtagttacaagctatatcaatgtcaaagtaaatgcacttcatcaaagctaagaagtcacaggaattgttctcaggtttttaaaaaaatttttcctgaattcaggaagtgtcttctgaatagcagctagccaaataaagcggtgtgtgtgtactgcagctgtaggtgaacttaaaaataataataaaaagaacaaataaagcagtgtgtaccagccgctgagccggccctgtctctgtcctgtgagacggaggggctgtgcctggaagtagaggggtgggcgttgctgcagcctgcagcccggcccgagtctgtcccagtgcctcctgtgtgcctggcacctcctcaggcatcttcctggctgccccatgttgcagctgagcctcagcgtcctgctagaactgcgtgtgggatttgctcactgccaggatgggactgtggctggaatggaggcagaaagaaacacatctcaaaggcaaaaatgacgcacgttgttgtctggaaaaatcttaagtg

SEQ ID NO: 40 - DNA, 1539 bp, Homo sapiens
  Gene sequence for Tieg2 T220M
atgcacacgccggacttcgcaggcccagacgacgcgcgcgcagttgacatcatggacatatgtgagtccatcctggagaggaagcggcatgacagcgaaaggtctacttgcagcatcttggagcagacagacatggaagctgtcgaggctcttgtttgtatgagctcctggggtcaaagatcccagaaaggtgacctgttgcggataagacccctcacgcctgtctctgactctggggatgtcaccaccactgtgcatatggatgcagccacacctgaactaccaaaagacttccattctttatcgactctgtgcataactcctcctcagagccctgatctcgtggagccatcgacaaggacacctgtttctccccaagtaacagattccaaagcatgtacagccacggatgttctccagtcctctgccgtagtggccagagctctgagcgggggcgcggagaggggcttgctgggtttggagccagtgcccagctctccctgcagggccaaggggactagcgtgatccgacacactggggagagccctgctgcctgctttcccaccatccagactccagattgccggctttctgacagcagagaaggagaagagcagcttctgggacactttgaaactttgcaggacacacacctcayggacagtttactcagcactaacttggtgtcctgtcagccctgcttgcacaagtctggtggcctgctgctcactgacaaaggccagcaggcagggtggcctggtgcagttcagacttgctcaccaaagaattatgaaaatgacctgcccaggaaaaccacccctctgatttctgtctctgtccctgctccccctgtcctttgccagatgatccctgtgactggacaaagtagcatgttaccagcttttttgaagccccctccccagttgtctgtggggactgtgagacccatcctagctcaggctgctccagcgcctcaacctgtgttcgtgggacctgctgtgcctcagggagctgtgatgttggtcctgccccagggagccctccctccgcctgccccctgtgcagccaatgtcatggctgccgggaataccaagttgttgccccttgcccctgctccagtgttcatcacctctagccaaaactgtgtccctcaggtagacttttcccgaaggaggaactatgtttgcagcttcccaggttgccggaagacctacttcaaaagttcccaccttaaggcccatcttcgcactcacacaggggagaagcctttcaactgcagctgggatggctgtgataaaaagtttgctcgttcggatgagctgtcacgccaccgcagaactcacacaggggagaagaagtttgtgtgcccggtgtgtgaccgacgtttcatgcgcagtgaccacctgacgaagcatgcccggcgccacatgacgaccaagaagatcccaggctggcaggcagaggttggcaagctgaacagaatcgcctctgcagagagcccggggagcccactggtgagcatgccagcctctgcctga

SEQ ID NO: 41 - DNA, 1539 bp, Homo sapiens
  Gene sequence for Tieg2 A347S
atgcacacgccggacttcgcaggcccagacgacgcgcgcgcagttgacatcatggacatatgtgagtccatcctggagaggaagcggcatgacagcgaaaggtctacttgcagcatcttggagcagacagacatggaagctgtcgaggctcttgtttgtatgagctcctggggtcaaagatcccagaaaggtgacctgttgcggataagacccctcacgcctgtctctgactctggggatgtcaccaccactgtgcatatggatgcagccacacctgaactaccaaaagacttccattctttatcgactctgtgcataactcctcctcagagccctgatctcgtggagccatcgacaaggacacctgtttctccccaagtaacagattccaaagcatgtacagccacggatgttctccagtcctctgccgtagtggccagagctctgagcgggggcgcggagaggggcttgctgggtttggagccagtgcccagctctccctgcagggccaaggggactagcgtgatccgacacactggggagagccctgctgcctgctttcccaccatccagactccagattgccggctttctgacagcagagaaggagaagagcagcttctgggacactttgaaactttgcaggacacacacctcacggacagtttactcagcactaacttggtgtcctgtcagccctgcttgcacaagtctggtggcctgctgctcactgacaaaggccagcaggcagggtggcctggtgcagttcagacttgctcaccaaagaattatgaaaatgacctgcccaggaaaaccacccctctgatttctgtctctgtccctgctccccctgtcctttgccagatgatccctgtgactggacaaagtagcatgttaccagcttttttgaagccccctccccagttgtctgtggggactgtgagacccatcctagctcaggctgctccagcgcctcaacctgtgttcgtgggacctgctgtgcctcagggagctgtgatgttggtcctgccccagggakccctccctccgcctgccccctgtgcagccaatgtcatggctgccgggaataccaagttgttgccccttgcccctgctccagtgttcatcacctctagccaaaactgtgtccctcaggtagacttttcccgaaggaggaactatgtttgcagcttcccaggttgccggaagacctacttcaaaagttcccaccttaaggcccatcttcgcactcacacaggggagaagcctttcaactgcagctgggatggctgtgataaaaagtttgctcgttcggatgagctgtcacgccaccgcagaactcacacaggggagaagaagtttgtgtgcccggtgtgtgaccgacgtttcatgcgcagtgaccacctgacgaagcatgcccggcgccacatgacgaccaagaagatcccaggctggcaggcagaggttggcaagctgaacagaatcgcctctgcagagagcccggggagcccactggtgagcatgccagcctctgcctga

SEQ ID NO: 42 - DNA, 21 bp, Artificial
  Description of Artificial sequence Primer
gacacacacctcayggacagt

SEQ ID NO: 43 - DNA, 30 bp, Artificial
  Description of Artificial sequence Primer
ttggtcctgccccagggakccctccctccg

SEQ ID NO: 44 - protein, 512 aa, Homo sapiens
  misc_feature (220)..(220): The ′Xaa′ at location 220 stands for Thr, or Met.
MHTPDFAGPDDARAVDIMDICESILERKRHDSERSTCSILEQTDMEAVEALVCMSSWGQRSQKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLXDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGALPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

SEQ ID NO: 45 - protein, 512 aa, Homo sapiens
  misc_feature (347)..(347): The ′Xaa′ at location 347 stands for Ala, or Ser.
MHTPDFAGPDDARAVDIMDICESILERKRHDSERSTCSILEQTDMEAVEALVCMSSWGQRSQKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLTDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGXLPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

(19) United States
(12) Patent Application Publication
(10) Pub. No.: US 2006/0223063 A1
(43) Pub. Date: Oct. 5, 2006

(54) Method of diagnosis of type 2 diabetes and early onset thereof

(76) Inventors: Philippe Froguel, Bagnolet (FR); Christian Dina, Paris (FR); Bernadette Paulina Neve, Menen (BE); Danielle Melloul, Ashkelon (IL)

Correspondence Address: SUGHRUE MION, PLLC, 2100 PENNSYLVANIA AVENUE, N.W., SUITE 800, WASHINGTON, DC, 20037, US

(21) Appl. No.: 10/546,451

(22) PCT Filed: Feb. 20, 2004
(86) PCT No.: PCT/IB04/00947

§ 371 Date: Jan. 4, 2006

(30) Foreign Application Priority Data
Feb. 20, 2003 (EP) 03290412.0

(51) Int. Cl. C12Q 1/68

(52) U.S. Cl. 435/6

(57) ABSTRACT

The invention pertains to the field of human genetics and relates to new methods of diagnosis and therapy of type 2 diabetes (T2D), early onset type 2 diabetes and Maturity-Onset Diabetes of the Young (MODY) in a human subject, based on the identification of the A185G variation located at exon 2 of the tieg2 gene as well as the G-1534C, 54a, 304a, +659 C>T (Thr220Met) and +1039 G>T (A1a347Ser) variations.

The invention pertains to the field of human genetics and relates to new methods of diagnosis and therapy of type 2 diabetes, early onset type 2 diabetes and Maturity-Onset Diabetes of the Young (MODY) in a human subject, based on the identification of the A185G variation located at exon 2 of the tieg2 gene as well as the G-1534C, 54a, 304a, +659 C>T (Thr220Met) and +1039 G>T (A1a347Ser) variations.

Type 2 diabetes, also known as noninsulin dependent diabetes mellitus (NIDDM) or adult onset diabetes, is the most common form of diabetes. In type 2 diabetes, the pancreatic cells produce little or no insulin, or the cells throughout the body are unable to utilize the insulin (insulin resistance). It represents 95% of all cases in the western countries, which affects about 15 million people in the United States alone.

As of today, the fasting plasma glucose (FPG) test is employed for diagnosing diabetes. However, this test is only relevant once the disease has developed. Therefore, there is a need for a diagnostic test capable of evaluating the genetic risk factor associated with this disease. Such a test would be of great interest in order to adapt the lifestyle of people at risk and to prevent the onset of the disease.

There is strong evidence to support the involvement of genetic factors in the genesis of type 2 diabetes. For example, the risk rate for type 2 diabetes between identical twins is around 60 to 90 percent (Bennett P H. Epidemiology of diabetes mellitus. In: Rifkin H, Porte D Jr., eds. Ellenburg and Rifkin's Diabetes Mellitus. New York: Elsevier, 1990: 363-77). In addition, having one first-degree relative with type 2 diabetes doubles the risk of developing the disease (Leslie R D, Pyke D A. Genetics of diabetes. In: Alberti K G, Krall L P, eds. The diabetes annual. Amsterdam: Elsevier, 1987: 39-54).

More recently, numerous scientific studies have established that there is a genetic predisposition to type 2 diabetes. We have performed a genome wide scan of diabetic and obese families, which led to the identification of several genomic regions showing evidence for linkage to these diseases (Vionnet N et al. (2000): Genomewide search for type 2 diabetes susceptibility genes in French whites: Evidence for a novel susceptibility locus for early onset diabetes on chromosome 3q27 qter and independent replication of a type 2 diabetes locus on chromosome 1q21 q24. Am. J. Hum. Genet. 67: 1470 1480 and Hager J et al. (1998): A genome wide scan for human obesity genes reveals a major susceptibility locus on chromosome 10. Nat. Genet. 20 : 304 308.).

Further fine mapping with microsatellite markers allowed us to limit this region to approximately 5 Mbases, which makes it feasible to identify candidate genes and apply a LD mapping strategy for this region. LD mapping involves the genotyping of a vast amount of single nucleotide polymorphisms (SNPs).

Over the years, we have developed complete strategies and methodologies to identify positional gene candidates. These include statistical analysis, a bioinformatic pipeline, SNP genotyping and tissue expression profiling. Our approach has resulted in fine mapping of the chr2 interval and analysis of this genomic region by a positional cloning approach. The SNP map established for chromosome 2 region has lead to the identification of TIEG2 as gene related to susceptibility to type 2 diabetes.

Both TIEG genes are induced by TGF-β (Cook et al. 1998; Blok et al. 1995) and may control pancreatic cell growth (Cook et al. 1998; Cook and Urrutia 2000). TIEG1 over expression in the exocrine PANC1 cell line induced apoptosis (Tachibana et al. 1997) and a similar proliferation inhibiting effect was shown for TIEG2 (Cook et al. 1998). Transgenic mice that express TIEG2 in the acinar cells of the exocrine pancreas (Mladek et al. 1999) show a similar pancreatic atrophy as mice over expressing TGF-β (Sanvito et al. 1995). Moreover, TIEG1 has been shown to induce similar effects as TGF-β (in osteoblast cells (Hefferan et al. 2000).

Thus, TGF-β induced TIEG 1 and 2 are able to mimic effects of TGF-β, in particular those effects observed on exocrine pancreatic tissues.

In connection with the invention, we identified and analyzed the variant A185G located at exon 2 of TIEG2 (=2SNP199b, TG2Q62R, TG2A185G) and we have shown that TIEG2 is related to susceptibility to type 2 diabetes (T2D). The SNP199b (A>G) is a non synonymous (G1n62Arg) variation in exon 2 of TIEG2, which can alter the repressive properties of TIEG2 on transcription. In connection with the invention, we identified and analyzed an additional variant, G-1534C, which is in close disequilibrium with A185G. This variant is located 1.5 Kb from the translation start site and may reside in the yet undefined promotor of the TIEG2 gene. Analysing this sequence with TransFac (Heinemeyer et al 1998) predicted that the introduction of the C allele creates a AP-1 site. The presence of both the -1534C and 185G allele could result in overexpression of a non-functional TIEG2 protein. We have also discovered that rare mutants in TIEG2 are related to Maturity-Onset Diabetes of the Young (MODY) and early onset T2D.

In this regards, altered regulation of progenitor cells in the developing pancreas and/or altered neogenesis of β cells in the adult human pancreas, which might be caused by a less functional TIEG2 regulation, may change the balance between exocrine and endocrine cells.

TIEG2 is implicated in the endocrine exocrine balance of the pancreas, and is postulated here to be important in the development and regeneration of pancreatic tissues, which could influence the development of type 2 diabetes. TIEG2 is ubiquitous expression, but has elevated mRNA levels in skeletal muscle, exocrine pancreas, brain and adipose tissue, which suggest that TIEG2 may have additional roles, which could involve proliferation/differentiation and cell cycle regulation, that could influence susceptibility to type 2 diabetes in several tissues.

### DESCRIPTION

Therefore, the present invention contributes to a better handling of type 2 diabetes and provides evidence of the implication of TIEG2 mediated signaling in susceptibility for type 2 diabetes. As a result, TIEG2 represents a novel target for drug design for Type 2 diabetes. The association of TIEG2 variant TG2Q62R with type 2 diabetes is particularly useful as a diagnostic test to analyze predisposition to type 2 diabetes.

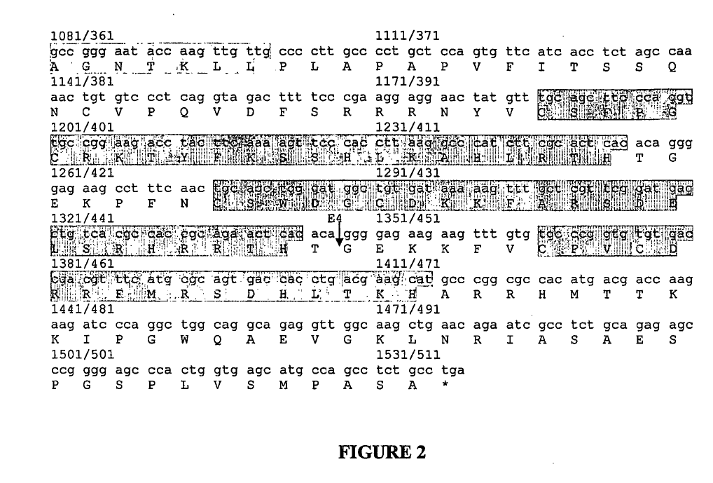

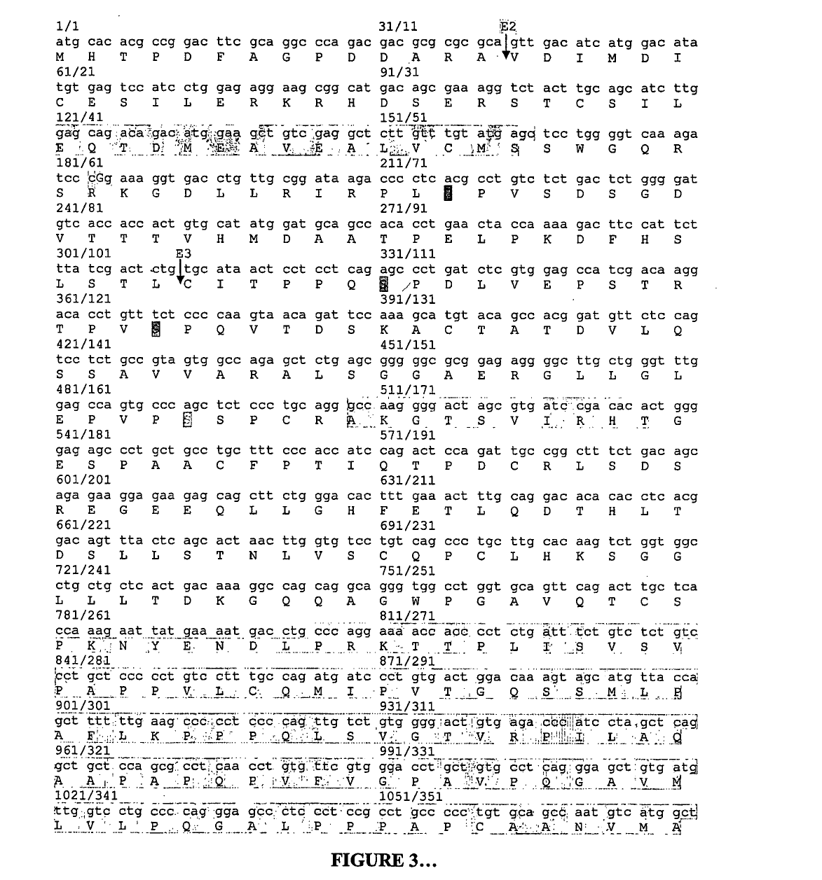

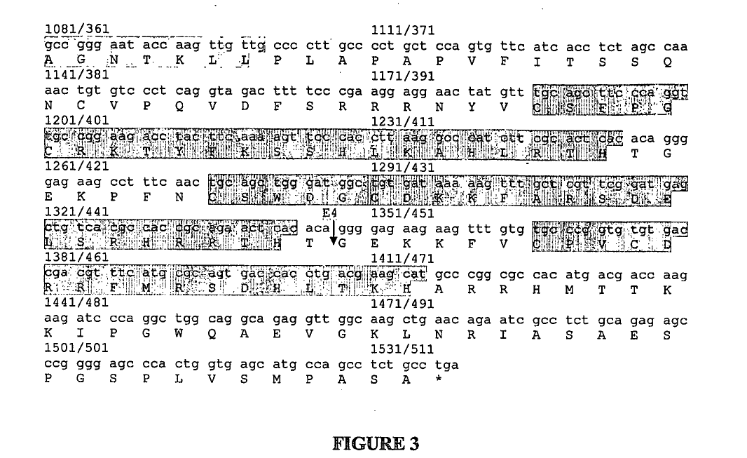

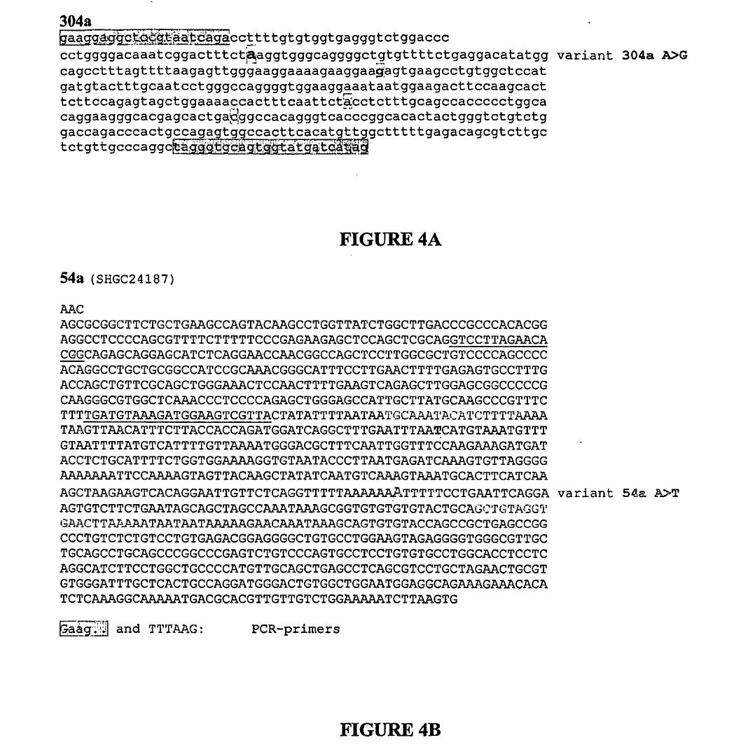

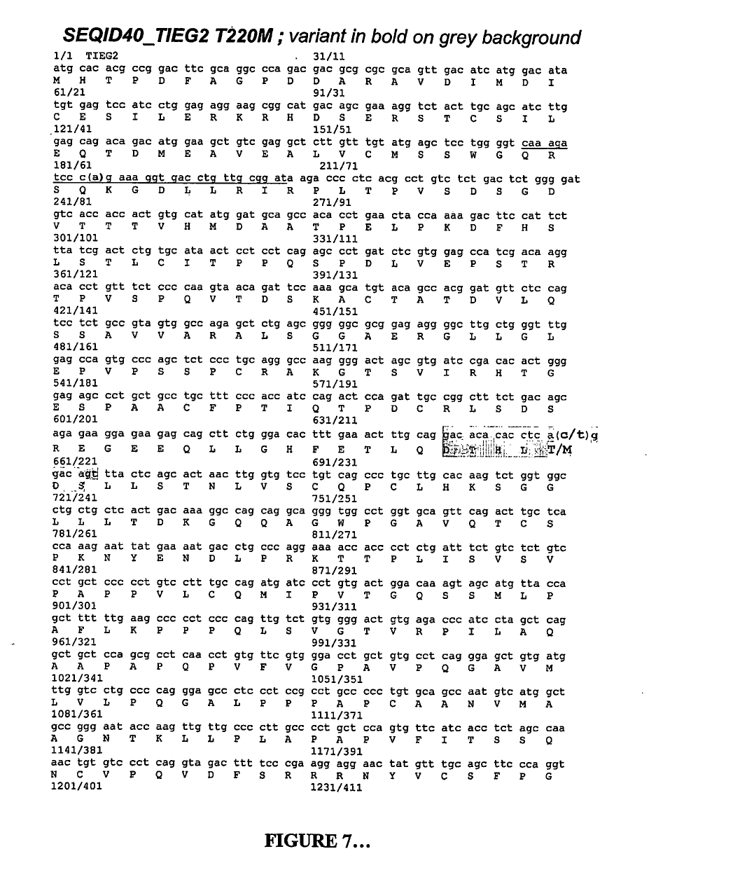

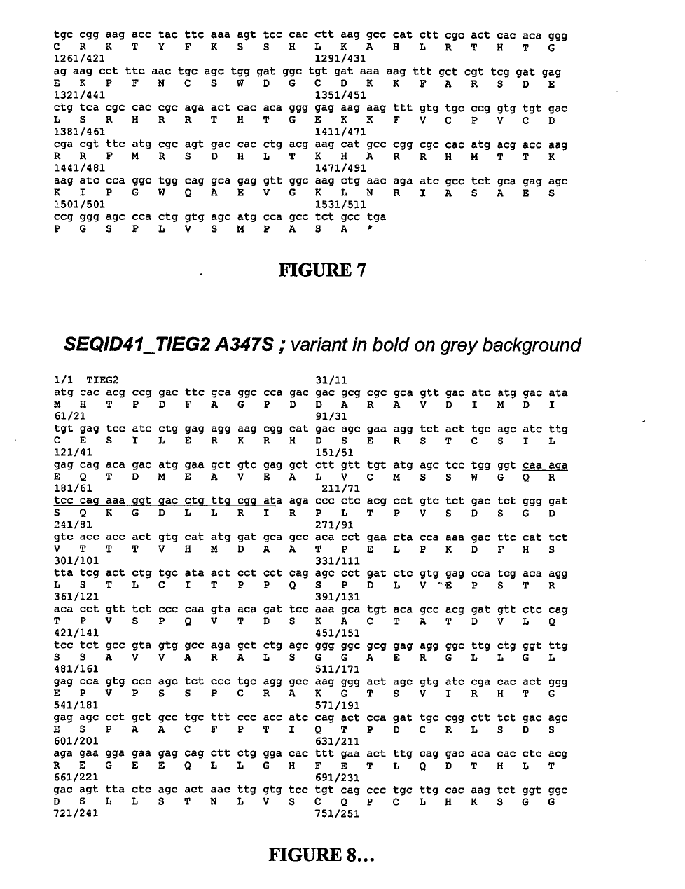

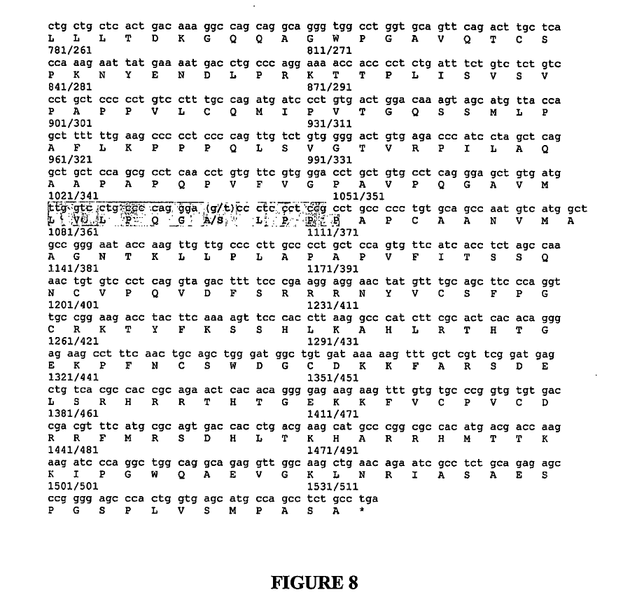

In a first aspect, the invention relates to a method for diagnosing a predisposition for type 2 diabetes, early onset type 2 diabetes and Maturity-Onset Diabetes of the Young (MODY) in a human subject which comprises determining whether there is a germline variation in the sequence of the tieg2 gene, said variation being indicative of a predisposition to type 2 diabetes, wherein said variation is selected from A185G numbered with regards to the start codon of the tieg2 gene, the coding sequence of which is represented by SEQ ID No 1, G-1534C as shown in SEQ ID No 4, 54a or 304a as shown in FIGS. 4A and 4B, and +659 C>T (Thr220Met) and +1039 G>T (A1a347Ser) of which the sequences are represented in SEQ ID No6 and 7 as shown in FIGS. 7 and 8 respectively.

It also relates to a method for diagnosing a predisposition for type 2 diabetes as depicted above comprising determination of the presence of germline variations that are in close association with a variation selected from variation A185G, G-1534C, 54a or 304a, +659 C>T (Thr220Met) and +1039 G>T (A1a347Ser).

The invention relates more generally to the study of the tieg2 gene. In this regard, the invention relates to a method for identifying variations in the sequence of the tieg2 gene, comprising determining whether there is a mismatch between molecules (1) a region tieg2 gene genomic DNA isolated from said sample, and (2) a nucleic acid probe complementary to human wild-type region of the tieg2 gene DNA, when molecules (1) and (2) are hybridized to each other to form a duplex, and said mismatch being due to the presence of at least one variation in the sequence of the tieg2 gene genomic DNA isolated from said sample.

More specifically, the invention is aimed at a method for identifying variations in exon 2 of the tieg2 gene comprising determining whether there is a mismatch between molecules (1) exon 2 of the tieg2 gene genomic DNA isolated from said sample, and (2) a nucleic acid probe complementary to human wild-type exon 2 region of the tieg2 gene DNA as represented by SEQ ID No 2, when molecules (1) and (2) are hybridized to each other to form a duplex, and said mismatch being due to the presence of at least one variation in the sequence of the tieg2 gene genomic DNA isolated from said sample.

In the above mentioned method, mRNA of the sample is contacted with a tieg2 gene probe under conditions suitable for hybridization of said probe to a RNA corresponding to said tieg2 gene and hybridization of said probe is determined, and the level of signal after hybridization is compared with a standard signal (which is either a positive control, a negative control or both).

The hybridization complex emits a signal, which may be due to the labeling of the probe, or directly of the mRNA. The different labels that may be used are well known to the person skilled in the art, and one can cite ³²P, ³³P, ³⁵S, ³H or ¹²⁵I. Non radioactive labels may be selected from ligants as biotin, avidin, streptavidin, dioxygenin, haptens, dyes, luminescent agents like radioluminescent, chemoluminescent, bioluminescent, fluorescent or phosphorescent agents. In order to identify the SNPs mentioned in the present application, it is possible to contact a tieg2 gene probe with genomic DNA isolated from said sample under conditions suitable for hybridization of said probe to said gene and hybridization of said probe is determined.

Said tieg2 gene region probe is either a “wild-type” DNA, (i.e. the searched SNP is not present on the probe, and hybridization occurs when no SNP according to the invention is present on the DNA in the sample) or a “mutant” one (i.e. carrying the searched SNP, and hybridization only occurs if the SNP is present on the DNA in the sample).

The person skilled in the art knows the techniques to determine the allele specific probes to use in this embodiment, their lengths, or the hybridization conditions.

In another embodiment, the invention is performed by determining whether there is an alteration in the germline sequence of the tieg2 gene exon 2 region or other regions in said sample by observing shifts in electrophoretic mobility of single-stranded DNA from said sample on denaturing or non-denaturing polyacrylamide gels. Said single-stranded nucleic acids may be obtained after amplification of the genomic DNA, using suitable primers, and denaturation (the gel and electrophoresis conditions are usually denaturing for such a purpose).

In another embodiment, the invention is performed by amplification of all or part of the tieg2 gene exon 2 region or other regions from said sample, and determination of the sequence of said amplified DNA.

In another embodiment, allele specific oligonucleotide primers are employed to determine whether a specific tieg2 mutant allele is present in said sample, the amplification only occurring in this case.

For example, these primers cover the sequences -caaagatcccRgaaaggtgac-(SEQ ID No 5) and -ggggtgctgastgggaagagg-(SEQ ID No 6) for, respectively, A185G and G-1534C, wherein R designates A or G and wherein S designates C or G.

The invention is also directed to a method for identifying variations in the tieg2 gene comprising determining whether there is a mismatch between molecules (1) the tieg2 gene genomic DNA isolated from said sample, and (2) a nucleic acid probe complementary to human tieg2 gene DNA covering the +659 C>T (Thr220Met) and +1039 G>T (A1a347Ser) variants sequences, such as a probe deriving from the sequence gacacacacctcaxggacagt-(SEQ ID No 42) wherein X is C or T and a probe deriving from the sequence ttggtcctgccccagggaYccctccctccg-(SEQ ID No 43) wherein Y is G or T.

In this regards, the invention is aimed at Primers covering the +659 C>T (Thr220Met) and +1039 G>T (A1a347Ser) variants sequences. For example a primer for the +659 C>T variant can encompasses or be derived from the sequence -pacacacacctXggacagt-(SEQ ID No 42) wherein X is C or T. A primer for the +1039 G>T variant can encompasses or be derived from the sequence -ttggtcctgccccagggaYccctccctccg-(SEQ ID No 43) wherein Y is G or T.

In another embodiment, all or part of the tieg2 gene exon 2 region from said sample is cloned to produce a cloned sequence and the sequence of said cloned sequence is determined.

The method depicted above can be practiced by amplification of tieg2 gene exon 2 region sequences in said sample and hybridization of the amplified sequences to one or more nucleic acid probes issued from the wild-type tieg2 gene exon 2 region sequence (as represented in SEQ ID No 2) or a mutant tieg2 gene exon 2 region sequence, said probes being selected from probes specific for the A185G variation and the G-1534C variation.

The A185G variation assay can be performed by a PCR with oligonucleotide primers to amplify the genomic sequence of exon 2 TIEG2 5′-CTC GGT GTT TGT TGC TAT AGA CT-3′ (SEQ ID No 7) and 5′-CAG GGA ATC TTC TCA CAA GTT CT-3′ (SEQ ID No 8), Table 1), whereafter the allelic variation can be measured by differential Tm changes using a lightcycler (Roche) with the probes LCRed640-ATC CCA GAA AGG TGA CCT (SEQ ID No 33) and TCT TGT TTG TAT GAG CTC CTG GGG TCA-fluorescine (SEQ ID No 34).

The G-1534C variation assay can be performed by a PCR with oligonucleotide primers to amplify the genomic sequence upstream of TIEG2 (5′-GTT TAA GAC GAA AGA ACC GTG ATA-3′ (SEQ ID No 9) and 5′-GAA CAG CAA GTG CGA GGA C-3′, (SEQ ID No 10), Table 1), whereafter the allelic variation can be measured by differential Tm changes using a lightcycler (Roche) with the probes LCRed640-TCTTCCCACTCAGCAC(SEQ ID No 35) and TCCCTGCGTCCACTCCAGCTCCCAGA-fluorescine (SEQ ID No 36).

The fluorescent molecules attached to the probes are shown as examples and are not limiting.

In still another embodiment, the invention is aimed at a probes or a primer selected from SEQ ID No 5 to SEQ ID No 36 (see Tables 1 hereinafter).

TABLE 1
TIEG2 primers
promoter region (1.8 Kb)

| | | | | |
|---|---|---|---|---|
| 2SNP437 | GTT TAA GAC GAA AGA ACC GTG ATA | GAA CAG CAA GTG CGA GGA C | 399 | A: 85Ins(actta); |
| | SEQ ID No 9 | SEQ ID No 10 | | B: 192(g/c) = G |
| | | | | 1534C |
| 2SNP436 | GGA ACC ATG CTG TTT ATT TTC GTC | GTC TTC ATG CGA GCA GTT TAT CAC | 411 | A: 181Ins(AG), |
| | SEQ ID No 11 | SEQ ID No 12 | | B: 248(C/T) |
| 2SNP442 | GAG ACG CGA GCA ACC GAA ATA | AGA GGG GAC CAC GCT TAT AGG AAC 3′ | 476 | A: 24Ins(g), B: 63(T/A) |
| | SEQ ID No 13 | SEQ ID No 14 | | |
| 2SNP453 | aaac aaccaaacaa aagccagg | 3′-gg tgtattttggttcgcctccg | 442 | |
| | SEQ ID No 15 | SEQ ID No 16 | | |

Exon 1

| | | | |
|---|---|---|---|
| 2SNP451 | gggcacgaa ttggctggtg gtgg | 3′-g cgggactactcgtctgagcg | 369 |
| | SEQ ID No 17 | SEQ ID No 18 | |
| 2SNP444 | TTC ATA TGC GTC TTG GTC AT | CGC TCA GAC GAG TAG TCC C 3′ | 355 |
| | SEQ ID No 19 | SEQ ID No 20 | |

Exon 2

| | | | | |
|---|---|---|---|---|
| 2SNP199 | CTC GGT GTT TGT TGC TAT AGA CT | CAG GGA ATC TTC TCA CAA GTT CT | 500 | A: 40(C/T), |
| | SEQ ID No 7 | SEQ ID No 8 | | B: 320(A/G) = A185G, |
| | | | | Q62R |

Exon 3

| | | | | |
|---|---|---|---|---|
| 2SNP417 | ACA GGT GTC CTT GTC GAT GG | AGA AAG CAG ATT TGG AGA GGG ATA | 373 | A: B4deI(A), B: 317(G/A) |
| | SEQ ID No 21 | SEQ ID No 22 | | |
| 2SNP305 | ATC TCG TGG AGC CAT CG | ACA GAA ATC AGA GGG GTG GT | 496 | 326(C/T) |
| | SEQ ID No 23 | SEQ ID No 24 | | |
| 2SNP306 | GGC CTG GTG CAG TTC AGA | GAA GAT GGG CCT TAA GGT GG | 492 | 432(A/T) |
| | SEQ ID No 25 | SEQ ID No 26 | | |
| 2SNP416 | ACG TCT ACT GCA ATG TGA ACA GA | CCT ACT TCA AAA GTT CCC ACC TTA | 354 | No variations identified |
| | SEQ ID No 27 | SEQ ID No 28 | | |

Exon 4

| | | | | |
|---|---|---|---|---|
| 2SNP300 | TTT CCT TTC TTA ATA TGT A | GAG GAA TCC ATC AGT TC 3 | 391 | No variations identified |
| | SEQ ID No 29 | SEQ ID No 30 | | |
| 2SNP418 | AAA AGA AGG CTG CAA AAT GAT CTA | CCT CTG CCT GAA AGG TCC AT | 398 | No variations |
| | SEQ ID No 31 | SEQ ID No 32 | | identified/polya |

The invention is also directed to the fragments of SEQ ID No 1 and SEQ ID No 4 amplified with the probes listed in table 1 and diagnostic kits comprising at least one of the identified variants.

The invention also relates to a method which comprises determining in situ hybridization of the tieg2 gene exon 2 region in said sample with one or more nucleic acid probes issued from a wild-type or a mutant tieg2 gene exon 2 region sequence. These methods includes CGH-fish or CGH arrays at the locus of the T

The inventors have demonstrated that the presence of the GIn62Arg variation of TIEG2 may interfere with the TIEG2 transcriptional repression activity in several ways. It is thus credible to speculate that other modifications in the exon 2 region or in other regions of the tieg2 gene, leading to a modification of the transcriptional repression activity of TIEG2 protein, will also lead to predisposition to type 2 diabetes, due to an alteration of the interaction with mSin3A.

In general, shown for the first time for Mad proteins, interaction with the corepressor mSin3A evokes recrutement of histone deacetylases and the formation of a functional transcription repressor complex. Recently it has been shown that phosphorylation nearby the first repressor domain disrupts the mSin3A binding to TIEG2 and results in loss of TIEG2's repressor activity (Ellenrieder et al 2002). Similar, occurence of the 62R variant may interfere with mSin3A binding changing the repressor activity of TIEG2. In addition, occurence of the 185G variant with the -1534C (%) variant, which introduces a possible transcriptional upregulation of TIEG2 via introduction of an AP-1 response site in TIEG2's promoter, could result in overexpression of a non-functional TEG2 protein.

Thus, in one embodiment, the invention relates to a method for diagnosing a predisposition for type 2 diabetes in a human subject, wherein the alteration of the interaction of TIEG2 protein with mSin3A and expression levels of TIEG2 mRNA in said sample is investigated. Alteration of interaction between protein and nucleic acid can be detected by any techniques known in the art. In a second aspect, the invention relates to the GIn62Arg variant of TIEG2 of SEQ ID No. 3, the Thr220Met variant of TIEG2 and the A1a347Ser variant of TIEG2. It also relates to a variant of TEG2 displaying at least 1, 2 or 3 of the following variations:
GIn62Arg
Thr220Met
A1a347Ser

In particular, these polypeptides may be detected by immunoblotting or immunocytochemistry.

Thus, the invention is aimed at antibodies specific for these TIEG2 variants, i-e antibodies capable of discriminating between normal TIEG2 and variant TIEG2, can be used to detect the presence or the absence of said variant in the sample of a patient. Immunological assays can be performed using standard knowledge in the art. These include Western blots, immunohistochemical assays and ELISA assays. The invention encompasses a diagnostic kit comprising such an antibody and reagents suitable for these tests.

The invention opens up a new area in the treatment and/or prevention of type 2 diabetes, especially for patient in families where genetic susceptibility is suspected.

Thus, the invention also relates to a method for screening compounds capable of preventing and/or treating type 2 diabetes which comprises: combining (i) a candidate compound and (ii) a TIEG2 polypeptide and determining the amount of binding of the TIEG2 polypeptide to said compound. Indeed, agonists of TIEG2 will reinstate its transcriptional repression activity and can be considered as useful drugs for treating type 2 diabetes, alone or in association with other treatments.

In this embodiment, the term “TIEG2” must be understood as including the GIn62Arg, Thr220Met and A1a347Ser variants as well as wild-type TIEG2.

The invention also relates to a pharmaceutical composition comprising a pharmaceutically acceptable excipient and a compound identified by a method according to the invention, and to the use of a compound identified by a method according to the invention for the manufacture of a drug intended to treat and/or prevent type 2 diabetes.

The invention also relates to a method for the therapy of type 2 diabetes comprising administration of the pharmaceutical composition of the invention.

In order to correct the defect due to the GIn62Arg, Thr220Met and A1a347Ser variants, another embodiment of the invention relates to a vector, more particularly a vector suitable for gene therapy, comprising the coding sequence for the wild-type TIEG2 operatively linked to a promoter allowing the expression of said coding sequence in human cells. Such vectors can be adapted for direct in vivo gene transfer and include viral vectors, such as adenovirus, retrovirus, herpes virus vectors, and adeno-associated virus (AAV). Nonviral vectors may also be used, including liposomes, molecular conjugates, polymers and other vectors. Direct injection into tissues, intravenous or intra-arterial administration, inhalation, or topical application and other routes of administration are known in the art.

The invention also relates to new animal models useful for studying type 2 diabetes, wherein said tieg2 variant is expressed in said animal. The methods to obtain animals according to the invention are known by the persons skilled in the art, and are useful in particular for testing some drugs that can be identified according to the methods of the invention. The methods include genetically modifying animals to provide a new line capable of germinal transmission of said tieg2 variant gene, chimeric animals, and animals which have been transfected with a vector expressing said tieg2 variant.

The insertion of the construct in the genome of the transgenic animal of the invention may be performed by methods well known by the artisan in the art, and can be either random or targeted. In a few words, the person skilled in the art will construct a vector containing the sequence to insert within the genome, and a selection marker (for example the gene coding for the protein that gives resistance to neomycine), and may have it enter in the Embryonic Stem (ES) cells of an animal. The cells are then selected with the selection marker, and incorporated into an embryo, for example by microinjection into a blastocyst, that can be harvested by perfusing the uterus of pregnant females. Reimplantation of the embryo and selection of the transformed animals, followed by potential back-crossing allow to obtain such transgenic animal. To obtain a “cleaner” animal, the selection marker gene may be excised by use of a site-specific recombinase, if flanked by the correct sequences.

The invention thus relates to a transgenic non-human mammal having integrated into its genome the nucleic acid sequence of the tieg2 variant gene or the coding sequence thereof, operatively linked to regulatory elements, leading to the expression of TIEG2 variant protein as described above; i-e GIn62Arg, Thr220Met and A1a347Ser or any combinations thereof.

The tieg2 sequence foreseen for the preceding embodiment may be a human tieg2 gene sequence introduced within the genome of the animal of the invention, or the endogenous tieg2 gene sequence the promoter of which has been modified in order to induce overexpression, so long as the variation is present in the sequence.

Furthermore, the invention also relates to a transgenic non-human mammal whose genome comprises a disruption of the endogenous tieg2 gene, as an animal model for testing links bewteen TIEG2 and type 2 diabetes and studying early onset of T2D. In particular, said disruption comprises the insertion of a selectable marker sequence. In particular, said disruption is a homozygous disruption, and said homozygous disruption results in a null mutation of the endogenous gene encoding TIEG2.

The mammal of the invention is preferably a rodent such as a mouse.

### LEGENDS

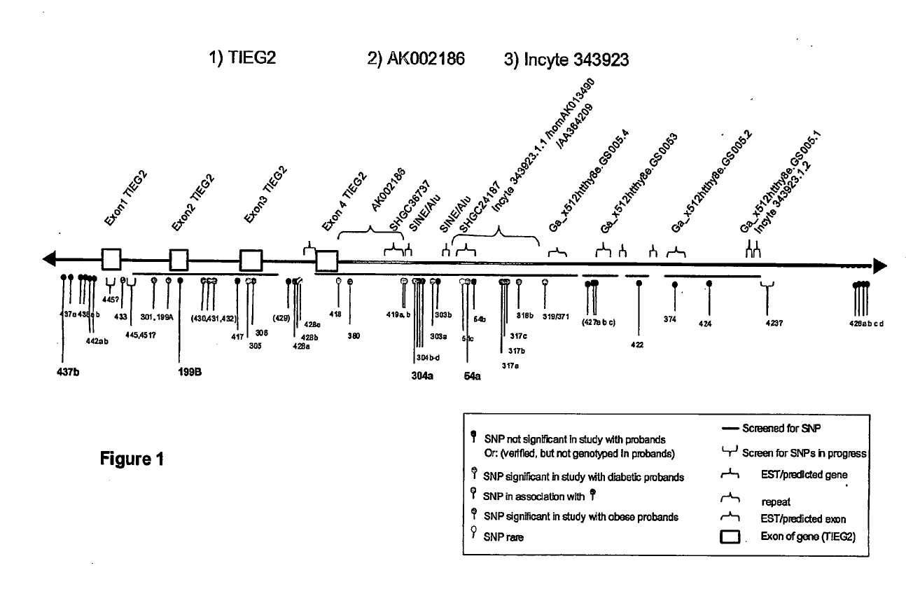

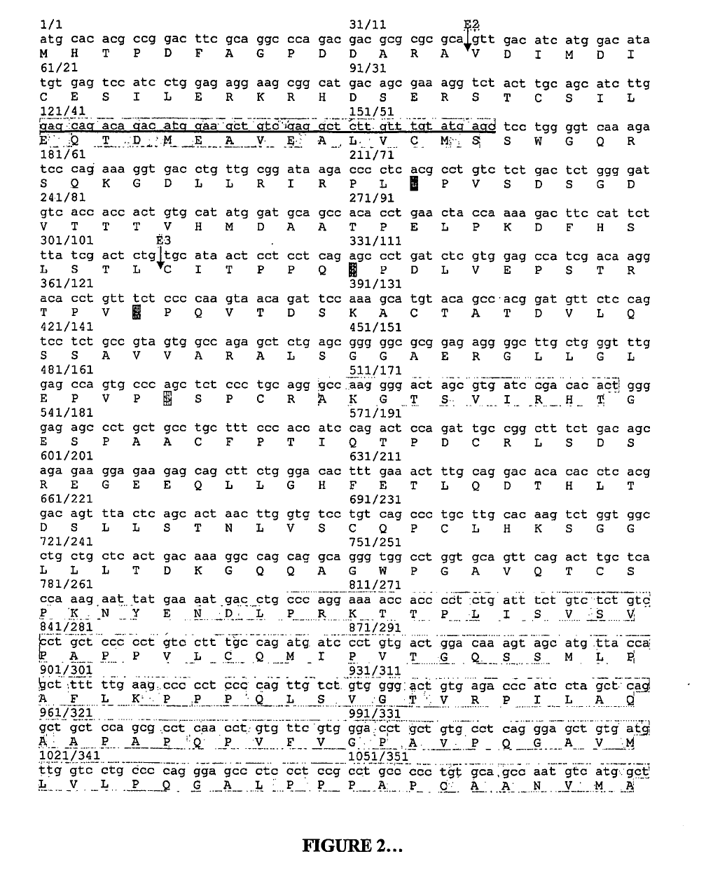

FIG. 1: SNP map and contig close to TIEG

[image]

[image]

FIG. 4: illustrated Sequence of 304a and 54a

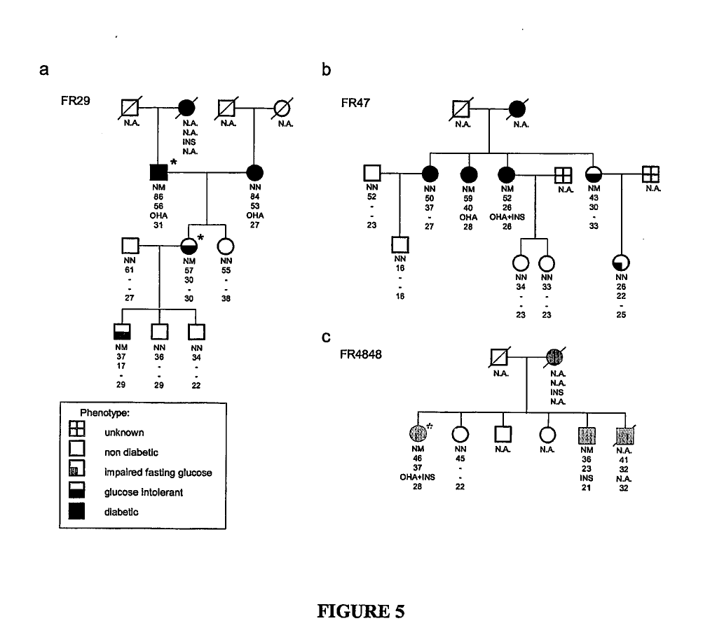

FIG. 5: characterization of TIEG2 variants. a) Pedigree of family with SNP +1039 G>T A1a347Ser (MODY-X FR29) and b,c) with SNP +659 C>T Thr220Met (early onset diabetes FR47 and FR4848). Astrisks indicate presence of diabetic complications (e.g. neuropathy). Underneath the symbols are genotype, age at medical examen, age of onset of diabetes/glucose intolerance, diabetic treatment (OHA; oral hypoglycaemic agent, INS; insulin) and BMI indicated.

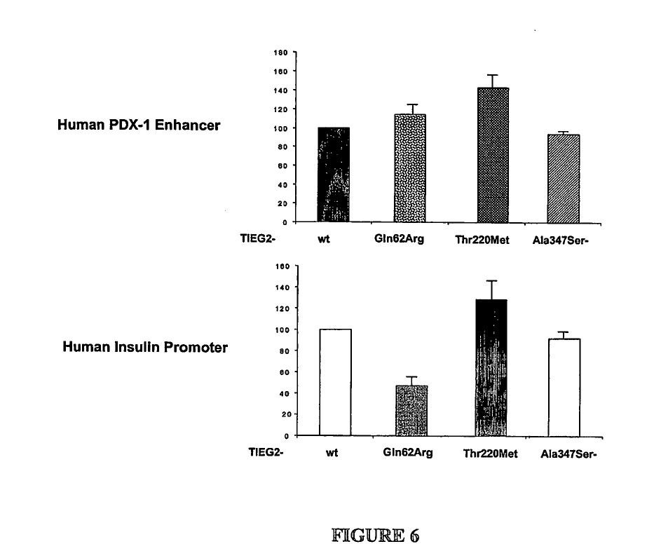

FIG. 6: variants described herein modify TIEG2 transcriptional potency Transfected TIEG2 expression vectors in CHO cells can activate the human PDX-1 enhancer and the Insuline promoter and construct containing SNP +185 A>G Gln62Arg, +1039 G>T A1a347Ser and SNP +659 C>T Thr220Met show that these variants modify TIEG2's transcriptional potency.

FIG. 7: illustrated Sequence SEQ ID No 40 c>t Thr220Met MODY/Early onset

FIG. 8: illustrated Sequence SEQ ID No 41 g>t A347S MODY f29

### EXAMPLE 1

### Genetic Evidence for Implication of TIEG2 in Type 2 Diabetes (T2D)

SNP199b (A>G) is a exonic variation of TIEG2. A robust association of SNP 199b (TG2Q62R) with T2D was shown to exist in a French Caucasian cohort of familial T2D and controls (Logistic regression (stratified for aga and sex) under dominant model TG2Q62R p<10-5). SNP 199b, 437b, 54a and 304a are in strong association (chi-square p<0.0001). SNP 437b (G>C) is a genomic variation that may reside in the yet undefined promotor of the TIEG2 gene. We have shown association of 54a with diabetes in all members of the genome wide scan families showing linkage evidence to chr 2 (phenotype large/strict, xtd lod-score 4.45 (p=0.04)). In contrast to the dominant association with familial type 2 diabetes (in the cohort of 352 controls vs 287 diabetics), no dominant association to diabetes was identified in the more heterogeneous ‘Corbeil’ population (947 diabetics). However, the variances remained recessively associated with a higher BMI.

The G1n62Arg is located in the proximity of the first repressor domain of the TIEG2-protein, which has been shown to interact with mSin3A corepressor. It has been shown that the R1 domain of TIEG2 adopts an a-helix conformation, and that this α-helical repression motif (oc-HRM), which is conserved in TIEG1, BTEB1, 3 and 4, is responsible for the interaction with mSin3A (Sin Interacting Domain; (Zhang et al. 2001)). Analyses of the secondary protein structure of TIEG2 (62Gln) and 62Arg-TIEG2 (protein containing TG2Q62R) with PIX (UK HGMP resource centre http://www.hizmp.mrc.ac.uk˜ showed that DSC (one out of three 2D analysing programs used (King et al. 2000)) predicted a prolonged α-helix for the 62Arg-TIEG2 in proximity of the SID. Thus, an altered conformation of 62Arg-TIEG2, may interfere with the α-HRM-mediated interaction with mSin3A.

In addition, the glutamine to arginine-change adds extra charge to the protein and may, therefore, change its isoelectric point and its binding properties to mSin3A.

Furthermore, G1n62Arg is next to a serine, which may be phosphorylated by Protein kinase C. Phosphorylation can play a crucial role in either regulating protein activity, translocation to the nucleus or interaction with other proteins and sensitivity to proteolytic degradation.

G-1534C is located 1.5 Kb from the translation start site and may reside in the yet undefined promotor of the TIEG2 gene. Analysing this sequence with TransFac (Heinemeyer et al 1998) predicted that the introduction of the C allele creates an AP-1 site. The presence of both the -1534C and 185G allele (linkage disequilibrium 0.95) could result in overexpression of a non-functional TIEG2 protein.

In summary, although TIEG2 has been implicated in the proliferation of exocrine pancreatic cells, analyses of the newly identified TG2Q62R (SNP199b) has, for the first time, directly implicated an altered TIEG2 in susceptibility for Type 2 diabetes. Furthermore, we hypothyse that since TIEG2 is ubiquitous expressed but higher expression levels are found in skeletal muscle, exocrine pancreas, brain and adypocytes, TIEG2 may affect susceptibility to T2D by its effect on these tissues. The association of TG2Q62R (SNP199b) with type 2 diabetes is useful as a diagnostic test to analyse predisposition to type 2 diabetes.

To further investigate the role of TIEG2 in the development of type 2 diabetes, we aimed at identifying target genes of TIEG2. First, we have concentrated on genes playing a major role in the pancreas. Based on the reported SP1-like binding site for TIEG2 (Cook et al 1998), we expect TIEG2 binding elements in the promoters of Smad4, IGF2 and PDX-1. For PDX-1 this binding element may reside in the recently characterised <Enhancer 1>element (Ben-Shushan et al 2001). Indeed, we have shown that a TIEG2 expression vector stimulates the activity of the PDX-1 enhancer element several fold, whereas it had no effects on the mutated enhancer element. PDX-1 is critical for development of the pancreas and regulates transcription of genes crucial for proper islet cell function, e.g. insulin gene (reviewed by Hui et al 2002). Moreover, mutations of the PDX-1 gene are held responsible for the development of maturity onset diabetes of the young type 4 (MODY 4). Thus, regulation of PDX-1 by TIEG2 shows that improper function of

TIEG2 may contribute to the development of type 2 diabetes. We are currently investigating the exact mechanism of PDX-1 gene regulation by TIEG2. On one hand, this may occur via direct interaction with the Enhancer 1 element. On the other hand, TIEG2 may affect PDX-1 gene expression via regulation of smad signalling since it was recently reported that the closely related TIEG1 regulate both smad7 and smad2 gene expression (Johnsen et al 2002a, b).

Therefore, we conclude that the G-1534C and Gln62Arg variation of TIEG2 interfere with TIEG2 transcriptional activity in several ways, and consequently have a causal association with the development of type 2 diabetes.

### EXAMPLE 2

### New Methods of Diagnosis and Therapy of Maturity-Onset Diabetes of the Young (MODY) and Early-Onset Type 2 Diabetes (T2D) in Relation to Rare TIEG2 Mutants

We have screened for TIEG2 mutants in 19 probands of French MODY families for which the underlying genetic cause was not known and in 171 probands with a diabetic onset before the age of 40 and minimal one affected first-degree relative (Male/Female 96/75, BMI 24.9±0.3 kg/cm2, age of 49.7±1.0 years, fasting glucose level of 8.9±0.3 mM, age of diabetic onset 32.6±0.5 years). We identified two rare mutants that were absent in 313 T2D patients and 313 control subjects. Mutant +1039 G>T (A1a437Ser) was found in a MODY-X family of four generation (FR29) and was transmitted with diabetes/glucose intolerance in the three analysed generations (FIG. 5a). The two spouses of the heterozygous carriers (of which one was diabetic too) and three non-diabetic siblings did not carry the variant. A1a437Ser is located within the third repressor domain of TIEG2 and bioinformatic analysis predicted it alters the secondary protein structure of this domain (PIX and SOPM). The second mutant, +659 C>T (Thr220Met) was present in two early onset T2D families. In family FR47 (FIG. 5b) three out of four sisters with glucose intolerance or frank diabetes were heterozygous carriers. The mutant was absent in five non-diabetic subjects analysed in this family. Families with early onset of diabetes are not per definition monogenic, and the fact that Thr220Met was absent in one diabetic family member is not unexpected in the context of a complex trait, and is in line with our previous conclusion that TIEG2 can influence T2D pathogenesis on a polygenic susceptibility background. In family FR4848 (FIG. 5c) the two diabetic subjects were heterozygous carriers and the non-diabetic subject was no carrier of Thr220Met Although located in between two repressor domains, 220Met-TIEG2 protein could have a prolonged α-helix structure in this region (PIX).

To analyse physiological consequences of TIEG2 impaired signalling pathway we investigated genes that might be regulated by TIEG2. We found that TIEG2 activates the enhancer E1 site of the PDX1/IPF1 gene, a key marker of pancreatic β-cell lineage and MODY4 gene, and the insulin gene promoter (FIG. 6). Since the β-cell dysfunction that proceeds the development of obesity-associated T2D is related to impaired compensatory increases in β-cell mass (reviewed by S. E Kahn, J. Clin. Endocrinol. Metab., 2001 and S. E. Kahn, Diabetologia 2003), any defect in PDX1 activity may have a dramatic effect on the development of diabetes.

Moreover, transfection experiments showed that insulin promoter activation by 62Arg-TIEG2 that contains the variant +185 A>G was impaired, which thus may unravel a novel mechanism causing genetic predisposition to diabetes.

### REFERENCES

Ben-Shushan E, Marshak S, Shoshkes M, Cerasi E, Melloul D. (2001) A pancreatic beta-cell-specific enhancer in the human PDX-1 gene is regulated by hepatocyte nuclear factor 3beta (HNF-3beta ), HNF-1alpha, and SPs transcription factors. J. Biol. Chem. 276, 17533-17540.
Blok L J, Grossmann M E, Perry J E, Tindall D J (1995) Characterization of an early growth response gene, which encodes a zinc finger transcription factor, potentially involved in cell cycle regulation. Mol Endocrinol 9: 1610-20.
Cook T, Gebelein B, Mesa K, Mladek A, Urrutia R (1998) Molecular cloning and characterization of TIEG2 reveals a new subfamily of transforming growth factor-beta-inducible Sp1-like zinc finger-encoding genes tnvolved in the regulation of cell growth. JBiol Chem 273: 25929-36.
Cook T, Urrutia R (2000) TIEG proteins join the Smads as TGF-beta-regulated transcription factors that control pancreatic cell growth. Am JPhysiol Gastrointest Liver Physiol 278: 6513-21.
Ellenrieder V, Zhang J S, Kaczynski J, Urrutia R. (2002) Signaling disrupts mSin3A binding to the Madl-like Sin3-interacting domain of TIEG2, an Sp1-like repressor. EMBO J. 21,2451-2460.
Hefferan T E, Reinholz G G, Rickard D J, Johnsen S A, Waters K M, Subramaniam M, Spelsberg T C (2000) Overexpression of a nuclear protein, TIEG, mimics transforming growth factor-beta action in human osteoblast cells. J Biol Chem 2 75: 20255-9.
Heinemeyer, T., Wingender, E., Reuter, I., Hermjakob, H., Kel, A. E., Kel, O. V., Ignatieva, E. V., Ananko, E. A., Podkolodnaya, O. A., Kolpakov, F. A., Podkolodny N. L. and Kolchanov, N. A.(1998) Databases on Transcriptional Regulation: TRANSFAC, TRRD, and COMPEL. Nucleic Acids Res. 26, 364-370
Hui H, Perfetti R.(2002) Pancreas duodenum homeobox-1 regulates pancreas development during embryogenesis and islet cell function in adulthood. Eur. J. Endocrinol. 146, 129-141.
Johnsen S A, Subramaniam M, Janknecht R, Spelsberg T C. (2002a) TGFbeta inducible early gene enhances TGFbeta/Smad-dependent transcriptional responses. Oncogene 21, 5783-5790.
Johnsen S A, Subramaniam M, Katagiri T, Janknecht R, Spelsberg T C. (2002b) Transcriptional regulation of Smad2 is required for enhancement of TGFbeta/Smad signaling by TGFbeta inducible early gene. J Cell Biochem. 87, 233-241.
Mladek A, Burgart J L, Urrutia R (1999) Morphological changes in the exocrine pancreas of transgenic mice overexpressing the TGFfi-inducible zinc finger transcription factor TIEG2 in an acinar-specific manner (abstract). Gastroenterology 116: G4837.
Sanvito F, Nichols A, Herrera P L, Huarte J, Wohlwend A, Vassalli J D, Orci L (1995) TGF-beta I overexpression in murine pancreas induces chronic pancreatitis and, together with TNF-alpha, triggers insulindependent diabetes. Biochem Biophys Res Commun 217: 1279-86.
Tachibana I, Imoto M, Adjei P N, Gores G J, Subramaniam M, Spelsberg T C, Urrutia R (1997) Overexpression of the TGFbeta-regulated zinc finger encoding gene, TIEG, induces apoptosis in pancreatic epithelial cells. J Clin Invest 99: 2365-74.

1. A method for diagnosing a predisposition for type 2 diabetes, early onset type 2 diabetes and Maturity-Onset Diabetes of the Young (MODY) in a human subject which comprises determining whether there is a germline variation in the sequence of the tieg2 gene, said variation being indicative of a predisposition to type 2 diabetes, wherein said variation is selected from A185G numbered with regards to the start codon of the tieg2 gene, the coding sequence of which is represented by SEQ ID No 1, G-1534C as shown in SEQ ID No 4, 54a or 304a as shown in FIGS. 4A and 4B, and +659 C>T (Thr220Met) and +1039 G>T (A1a347Ser) of which the sequences are represented in SEQ ID No40 and 41 as shown in FIGS. 7 and 8 respectively.

2. A method according to claim 1 comprising determination of the presence of germline variations that are in close association with a variation selected from variation A185G, G-1534C, 54a or 304a, +659 C>T (Thr220Met) and +1039 G>T (A1a347Ser).

3. A method for identifying variations in the sequence of the tieg2 gene, comprising determining whether there is a mismatch between molecules (1) a region tieg2 gene genomic DNA isolated from said sample, and (2) a nucleic acid probe complementary to human wild-type region of the tieg2 gene DNA, when molecules (1) and (2) are hybridized to each other to form a duplex, and said mismatch being due to the presence of at least one variation in the sequence of the tieg2 gene genomic DNA isolated from said sample.

4. A method for identifying variations in exon 2 of the tieg2 gene comprising determining whether there is a mismatch between molecules (1) exon 2 of the tieg2 gene genomic DNA isolated from said sample, and (2) a nucleic acid probe complementary to human wild-type exon 2 region of the tieg2 gene DNA as represented by SEQ ID No 2, when molecules (1) and (2) are hybridized to each other to form a duplex, and said mismatch being due to the presence of at least one variation in the sequence of the tieg2 gene genomic DNA isolated from said sample.

5. A method according to claim 4 comprising the amplification of tieg2 gene exon 2 region sequences in said sample and hybridization of the amplified sequences to one or more nucleic acid probes issued from the wild-type tieg2 gene exon 2 region sequence (as represented in SEQ ID No 2) or a mutant tieg2 gene exon 2 region sequence, said probes or primers covering the sequences -caaagatcccRgaaaggtgac-(SEQ ID No 5) and -ggggtgctgastgggaagagg-(SEQ ID No 6) for the TIEG2 variants A185G and G-1534C, respectively, wherein R designates A or G and wherein S designates C or G.

6. A method for identifying variations in the tieg2 gene comprising determining whether there is a mismatch between molecules (1) the tieg2 gene genomic DNA isolated from said sample, and (2) a nucleic acid probe complementary to human tieg2 gene DNA covering the +659 C>T (Thr220Met) and +1039 G>T (A1a347Ser) variants sequences such as a probe deriving from the sequence gacacacacctcaXggacagt-(SEQ ID No 42) wherein X is C or T and a probe deriving from the sequence ttggtcctgccccagggaYccctccctccg-(SEQ ID No 43) wherein Y is G or T.

7. A probes or a primer selected from SEQ ID No5 to SEQ ID No36 and primers deriving from SEQ ID No42 and 43.

8. A variant of TIEG2 selected from the GIn62Arg protein variant of TlEG2 of SEQ ID no 3, the Thr220Met protein variant, the A1a347Ser protein variant, and a variant displaying at least 2 of the following variations:
GIn62Arg,
Thr220Met,
A1a347Ser.

9. An antibody capable of discriminating between normal TIEG2 and the variant TIEG2 according to claim 8.

10. A diagnostic kit comprising an antibody according to claim 8 and reagents suitable for Western blots, immunohistochemical assays and ELISA assays or at least one probe according to claim 7.

11. A method for screening compounds capable of preventing and/or treating type 2 diabetes which comprises: combining (i) a candidate compound and (ii) a TIEG2 polypeptide and determining the amount of binding of the TIEG2 polypeptide to said compound.

12. A vector suitable for gene therapy, comprising the coding sequence for the wild-type TIEG2 operatively linked to a promoter allowing the expression of said coding sequence in human cells.

13. Use of a compound identified by the method of claim 11 or of a vector according to claim 11 to manufacture a medicament for preventing and/or treating type 2 diabetes.

14. An animal model useful for studying type 2 diabetes, wherein the protein variant of claim 8 is expressed in said animal.

15. A transgenic non-human mammal having integrated into its genome a nucleic acid sequence coding for protein variant of claim 8, operatively linked to regulatory elements, leading to the expression of a variant of TIEG2 selected from the GIn62Arg protein variant of TIEG2 of SEQ ID no 3, the Thr220Met protein variant, the A1a347Ser protein variant, and a variant displaying at least 2 of the following variations:
GIn62Arg,
Thr220Met,
A1a347Ser.

16. A transgenic non-human mammal whose genome comprises a disruption of the endogenous tieg2 gene, as an animal model for type 2 diabetes, early onset type 2 diabetes and Maturity-Onset Diabetes of the Young (MODY).

17. A transgenic non-human mammal according to claim 16 wherein said disruption is A185G, G-1534C, 54a, 304a, +659 C>T and +1039 G>T.
